(19) United States
(12) Patent Application Publication
(10) Pub. No.: US 2025/0009820 A1
(43) Pub. Date: Jan. 9, 2025

(54) WOMEN'S HEALTH PROBIOTIC FORMULATIONS AND METHODS OF USE THEREOF

(71) Applicant: NewGen 27, LLC d/b/a Doctor's Biome, Westbury, NY (US)

(72) Inventors: Howard F. Robins, Westbury, NY (US); A. Reza Kamarei, Princeton, NJ (US); Eric Finkelstein, Ridgefield, CT (US)

(21) Appl. No.: 18/763,204

(22) Filed: Jul. 3, 2024

Related U.S. Application Data
(60) Provisional application No. 63/525,456, filed on Jul. 7, 2023.

(51) Int. Cl. A61K 35/747; A61K 9/00; A61K 9/08; A61K 35/745; A61K 36/45; A61K 47/46

(52) U.S. Cl. CPC A61K 35/747; A61K 9/0095; A61K 9/08; A61K 35/745; A61K 36/45; A61K 47/46

(57) ABSTRACT

The present invention is directed to a novel women's health probiotic formulation and methods of using the women's health probiotic formulation that inhibits both E. coli and C. albicans. Probiotics of the invention utilize Bifidobacteria and Lactobacilli to formulate a novel women's health probiotic comprising cranberry powder effective against both pathogens.

### TECHNICAL FIELD OF THE INVENTION

The present invention generally refers to women's health probiotic formulations and methods to use the women's health probiotic formulations to inhibit the growth of harmful and pathogenic microorganisms.

### BACKGROUND OF THE INVENTION

Two of the most common medical problems in women are Urinary Tract Infections (UTIs) and Vaginal candidiasis (VC) also called vaginal yeast infections or Vulvovaginal candidiasis (VVC).

### Urinary Tract Infections

Urinary Tract Infection (UTI) is a common infection in any part of the urinary system (kidneys, ureters, bladder and urethra) affecting more than 3 million US cases per year. Most infections involve the lower urinary tract—the bladder and the urethra. Research shows that the risk for a UTI is as much as 14 times greater in women than in men. That risk increases with age, with women over the age of 55 having a greater chance of infection and reinfection than younger women. Uropathogens are able to invade the urinary tract and colonize in the bladder from touching contaminated surfaces. The vagina may act as a pool of the bacteria and result in self-infection, person to person spread, and contaminate food or water.

UTIs are now considered a severe public health problem as they are one of the top two most common bacterial infections world-wide, acquired at home, in hospitals and nursing homes. It is estimated that they account for 10-20% of all infections presenting in primary care facilities and between 30-40% of all bacterial infections treated in hospitals, creating a costly economic and personnel burden on the healthcare system. It is estimated that treatment costs for uncomplicated UTIs eclipse 6 billion USD worldwide. While sometimes self-limiting, they have a marked proclivity to reoccur.

Conventional treatments for UTIs include the use of antimicrobials. Non-antibiotic approaches and strategies such as the use of probiotics or cranberry have had minimal success both in prevention and treatment.

### Vaginal Candidiasis

Vaginal Candidiasis (VC) or VVC is an inflammation of the vagina that can result in discharge, itching and pain. The cause is usually a change in the balance of vaginal bacteria or an infection. There are three common types of vaginitis: bacterial vaginosis, yeast infections, and trichomoniasis. Yeast infections are usually caused by a naturally occurring yeast Candida albicans. Candida albicans (C. albicans) are a Gram-positive fungus and an opportunistic pathogenic yeast that is a common member of the human gut flora.

VVC affects millions of women every year. The estimated probability of being afflicted with VVC by age 50 varies widely by country as does the estimated probability of Recurrent Vulvovaginal Candidiasis (RVVC) with the likelihood of developing VVC increasing after the age of 55.

Conventional treatments for VVC use antifungals. However, although conventional antifungals can be effective against VVC, an increasing number of resistant strains have emerged, and adverse reactions are possible.

### SUMMARY OF THE INVENTION

The present invention is directed to a novel women's health probiotic formulation and methods of using the probiotic formulation that inhibits both E. coli and C. albicans. Probiotics of the invention utilize Bifidobacteria and Lactobacilli to formulate a novel probiotic, together with cranberry powder in fruit and liquid juices effective against both pathogens.

The present invention provides a women's health probiotic formulation comprising a liquid medium comprising liquid fruit and vegetable juice to which cranberry powder is added. After sterilizing the liquid medium a plurality of living probiotic bacterial strains of the genus Bifidobacterium and genus Lactobacillus in the fruit and vegetable juice medium, with the plurality of living probiotic bacterial strains of the genus Bifidobacterium including species B. bifidum, B. breve, B. lactis, B. longum, B. infantis and mixtures thereof and with the plurality of living probiotic bacterial strains of the genus Lactobacillus including species L. acidophilus, L. brevis, L. bulgaricus, L. casei, L. gasseri, L. paracasei, L. plantarum, L. reuteri, L. rhamnosus, L. salivarius and mixtures thereof. Each living bacterial strain is present in the liquid medium in substantially a same percentage.

The resultant formulation is a probiotic medium that includes a plurality of living bacterial strains belonging to the genus Bifidobacterium and genus Lactobacillus in a sterilized juice medium. The bacterial living strains of the genus Bifidobacterium include B. bifidum, B. breve, B. lactis, B. longum, B. infantis, and their mixtures. The living bacterial strains of the genus Lactobacillus include species L. acidophilus, L. brevis, L. bulgaricus, L. casei, L. gasseri, L. paracasei, L. plantarum, L. reuteri, L. rhamnosus, L. salivarius, and their mixtures. Each living bacterial strain is present in the liquid medium is present in substantially the same percentage. The probiotic formulation comprises a sugar content within predetermined concentration range of about 5% to about 15% (for example, 5%, 6%, 7%, 8%, 9%, 10%, 11%, 12%, 13%, 14%, or 15%).

The juice and vegetable medium of the probiotic formulation may be a sterilized liquid fruit and vegetable juice medium. The sterilized liquid fruit and vegetable juice medium may be at least one of or a mixture of mint, cucumber, apple, lettuce, kale, celery, and lemon. The sterilized liquid juice medium used in the probiotic formulation of the present invention is organic and devoid of pesticides, chemicals or additives of any sort. The liquid fruit and vegetable juice may comprise watermelon juice, apple juice, pomegranate juice, beet juice and strawberry juice

The cranberry powder may be present in the medium at a ratio of about 500 mg per 2 fluid ounces of liquid fruit and vegetable juice. The medium may be sterilized prior to addition of the living probiotic bacterial strains. The plurality of living probiotic bacterial strains is present in the medium may be at a ratio of 10-35 billion colony-forming units (CFU) per 2 fluid ounces of liquid fruit and vegetable juice. For example, the living probiotic bacterial strains may be added to the medium at a ratio of 10-35 billion colony-forming units (CFU) per 2 fluid ounces of liquid fruit and vegetable juice. The strains forming a part of the probiotic formulation of the first aspect of the invention may be actively living and pre-hydrated.

The probiotic formulation of the present invention may be in the form of a drinkable juice beverage which has a desirable shelf-life without the need of refrigeration, yet maintaining the viability of the bacterial strains. The probiotic formulation may also include a prebiotic.

Advantageously, probiotic formulations of the invention are useful in inhibiting the growth of E. coli and C. albicans, responsible for UTIs and VC respectively. Accordingly, aspects of the invention provide methods of improving the health of a suffering from an E. coli infection of the urinary tract with a formulation comprising a probiotic medium that includes a plurality of living bacterial strains belonging to the genus Bifidobacterium and genus Lactobacillus in a sterilized juice medium. The bacterial living strains of the genus Bifidobacterium include B. bifidum, B. breve, B. lactis, B. longum, B. infantis, and their mixtures. The living bacterial strains of the genus Lactobacillus include species L. acidophilus, L. brevis, L. bulgaricus, L. casei, L. gasseri, L. paracasei, L. plantarum, L. reuteri, L. rhamnosus, L. salivarius, and their mixtures. Each living bacterial strain is present in the liquid medium is present in substantially the same percentage. The probiotic formulation comprises a sugar content within predetermined concentration range of about 5% to about 15% (for example, 5%, 6%, 7%, 8%, 9%, 10%, 11%, 12%, 13%, 14%, or 15%).

Aspects of the invention also provide methods of improving the health of a woman suffering from a yeast infection with a formulation comprising a probiotic medium that includes a plurality of living bacterial strains belonging to the genus Bifidobacterium and genus Lactobacillus in a sterilized juice medium. The bacterial living strains of the genus Bifidobacterium include B. bifidum, B. breve, B. lactis, B. longum, B. infantis, and their mixtures. The living bacterial strains of the genus Lactobacillus include species L. acidophilus, L. brevis, L. bulgaricus, L. casei, L. gasseri, L. paracasei, L. plantarum, L. reuteri, L. rhamnosus, L. salivarius, and their mixtures. Each living bacterial strain is present in the liquid medium is present in substantially the same percentage. The probiotic formulation comprises a sugar content within predetermined concentration range of about 5% to about 15% (for example, 5%, 6%, 7%, 8%, 9%, 10%, 11%, 12%, 13%, 14%, or 15%).

Advantageously, probiotic formulations of the invention further provide a palatable taste for consumers, aiding in maintaining use of the probiotic formulations over time.

### BRIEF DESCRIPTION OF THE DRAWINGS

FIG. 1 is an image of E. coli a rod-shaped bacterium

FIG. 2 is an image of C. albicans visualized using scanning electron microscopy

FIG. 3 is an image of a women's health probiotic formulation of the invention.

FIG. 4 is an image of a women's health probiotic formulation of the invention in a falcon tube before (left) and after (right) centrifugation—4,500 RPM for 15 minutes.

FIG. 5 is an image of sorbitol agar plates with E. coli O157:H7 recovered from a control sample (1:10,000 dilution) and test sample (1:10 dilution)

FIG. 6 is an image of potato dextrose agar plates with C. albicans recovered from a control sample (1:10,000 dilution), and test sample (1:10 dilution)

FIG. 7 is a graph of the population of E. coli O157:H7 recovered from a test sample and Tryptic Soy Broth (control samples) incubated at 37° C.

FIG. 8 is a graph of the population of C. albicans O157:H7 recovered from a test sample and Tryptic Soy Broth (control samples) incubated at 37° C.

### DETAILED DESCRIPTION OF THE INVENTION

The present invention is directed to a novel women's health probiotic formulation and methods of using the women's health probiotic formulation that inhibits both E. coli and C. albicans. Probiotics of the invention utilize Bifidobacteria and Lactobacilli to formulate a novel probiotic comprising cranberry powder effective against both pathogens.

Prior to the present invention, both probiotics and cranberry products had been shown to separately be rather ineffective in treating UTIs and VV, with inconsistent results against in habiting either of E. coli or C. albicans. By the present invention, it was discovered that the combination of specific probiotic mixtures with cranberry products can effectively inhibit both E. coli and C. albicans.

### Probiotics

Probiotics are live micro-organisms that, when administered in adequate quantities, offer an array of health benefits to the host. The health benefits include reducing antibiotic-associated diarrhea, improving lactose intolerance, improving allergies, and treating infectious diarrhea. Probiotics available are marketed in several forms. For example, probiotics may be marketed in a dry form or in the form of a liquid. Probiotics in a dry form are not physiologically active. In order to make the dry probiotic cells physiologically active again, the probiotics are suspended in an aqueous environment in the presence of water and nutrients. The probiotic dry cells may acquire nutrition from a prebiotic. Prebiotics support probiotic bacteria by inducing their growth and activity. Prebiotics are compounds found in food that induce the growth of these beneficial probiotic micro-organisms. Dietary prebiotics are non-digestible fiber that stimulate and facilitate the growth of beneficial gut microbiota. On the other hand, probiotics in a liquid form are physiologically active as they are suspended in an aqueous medium, and have access to the nutrition present in the medium.

It is important to note that probiotics products are generally in the form of capsules (loose powder) or tablets (compressed powder). In their dry form, probiotic cells are in the state of “suspended animation” which means most of their vital functions are temporarily ceased. In other words, probiotic cells in dry form, while alive at the time of manufacturing and after freeze drying, are not living (not physiologically active). To make dry, alive probiotic cells become physiologically active, they need to be fully hydrated and surrounded in an aqueous environment with sufficient available water and nutrients. When probiotics are consumed in the form of a dry powder, they need to get fully hydrated in the highly acidic environment of the stomach (pH 1.5). When probiotics are consumed in the form of a liquid (e.g., in a blend of organic fruit and vegetable juices), they are already fully hydrated and suspended in an aqueous environment. In other words, such pre-hydrated probiotics are physiologically active and ready to function when consumed.

Among the wide range of probiotic micro-organisms, Bifidobacterium and Lactobacillus are recognized for being beneficial to gut health. Bifidobacterium species are Gram-positive, non-motile inhabitants of the human gastro-intestinal tract. Lactobacillus species constitute a substantial portion of the human gastro-intestinal system, allowing the gastro-intestinal to persist severe environments. Lactobacillus species are Gram-positive bacteria which exist in a synergistic relationship with the host, as they shield the host from potential pathogenic invasions, and in turn, receive nutrients from the host.

Accordingly, first aspect of the present invention is directed to a probiotic formulation for oral administration. The probiotic formulation of the present invention is a liquid juice medium comprising one or more strains of living probiotic bacteria. Any living probiotic bacteria suitable for human administration may be used. For example, probiotic bacterial strains may be selected from the genus Lactobacillus or Bifidobacterium. In one embodiment, the probiotic formulation may include a combination of such living bacterial strains. In one embodiment, the probiotic formulation may be a blend of living bacterial strains of B. bifidum, B. breve, B. lactis, B. longum, B. infantis, L. acidophilus, L. brevis, L. bulgaricus, L. casei, L. gasseri, L. paracasei, L. plantarum, L. reuteri, L. rhamnosus, L. salivarius, known as DOCTOR's BIOME SIGNATURE PROBIOTIC BLEND (DBSPB).—Each living bacterial strain present in the liquid juice medium is present in substantially the same percentage.

The probiotic formulation of the present invention may be in the form of a beverage suitable for oral administration. The probiotic formulation may be a dietary supplement. The beverage may comprise living bacterial strains dispersed in a liquid juice medium. The beverage may be in the form of a sterilized liquid fruit and vegetable juice medium. The sterilized liquid fruit and vegetable juice medium may include, but is not limited to, mint juice, cucumber juice, apple juice, lettuce juice, kale juice, celery juice, lemon juice, and mixtures thereof. The sterilized liquid fruit and vegetable juice medium may be organic and free of chemicals, pesticides, and/or additives. The probiotic formulation of the invention may include one or more prebiotics. Examples of prebiotics used in the probiotic formulation include but are not limited to inulin, maltodextrin, resistant starch, and/or mixtures thereof. The formulation is a drinkable juice beverage with a desirable shelf-life while also maintaining viability of the bacterial strains without the need of refrigeration or cooling of the beverage.

The probiotic formulation of the present invention may be formulated to include sugar at a predetermined concentration. In some embodiments, the probiotic formulation may comprise sugar in the range of 5%-15%. For example, the probiotic formulation may comprise sugar concentration of 5%, 6%, 7%, 8%, 9%, 10%, 11%, 12%, 13%, 14%, or 15%.

The probiotic formulation may further include excipients and/or additives, including but not limited to antioxidants and taste enhancers.

The probiotic formulation may contain living probiotic bacteria at a predetermined range of colony forming units. Colony forming units is a measure or a unit that provides an estimate of the number of viable microbial cells in a given sample.

Accordingly, the probiotic formulation may be formulated to provide between 1 billion and 200 billion colony forming units in a serving. The probiotic formulation may comprise between 1 billion and 200 billion colony forming units of pre-hydrated, actively living bacterial strains in the sterilized liquid fruit and vegetable juice medium. In one embodiment, there may be between 1 billion and 200 billion colony forming units of pre-hydrated, actively living bacterial strains in a 2-ounce bottle of the sterilized liquid fruit and vegetable juice medium.

### Cranberry Powder

Cranberry fruits or leaves have historically been used for bladder, stomach, and liver disorders, as well as diabetes, wounds, and other conditions. Today, cranberries are commonly promoted for the treatment of urinary tract infections.

However, cranberries have not been shown to be effective as a treatment for an existing UTI.

Some studies in people who are at increased risk for UTIs or those who have had recurrent UTIs have shown that cranberry products may decrease the risk of UTIs by about one-third. However, there remains uncertainty as to the effectiveness of cranberries for this use and skepticism as to the quality of research indicating such efficacy. Moreover, studies in certain populations at increased risk of UTIs, such as elderly people in long-term care and pregnant women, have had inconsistent results, and studies in other high-risk populations, such as women undergoing gynecological surgeries or people with multiple sclerosis, have not found cranberries to confer any benefit.

Cranberry products are generally thought to be safe. However, if consumed in very large amounts, they can cause stomach upset and diarrhea, particularly in young children. Little is known about whether it's safe to use cranberry for health purposes during pregnancy or while breastfeeding and there is conflicting evidence about whether cranberry interacts with the anticoagulant (blood thinner) warfarin.

On Jul. 21, 2020, the U.S. Food and Drug Administration announced in a “letter of enforcement discretion that it does not intend to object to the use of certain qualified health claims regarding consuming certain cranberry products and a reduced risk of recurrent urinary tract infection (UTI) in healthy women.” However, the FDA's letter of enforcement discretion reiterated that:
“Limited scientific evidence shows that by consuming 500 mg each day of cranberry dietary supplement, healthy women who have had a urinary tract infection (UTI) may reduce their risk of recurrent UTI.”
“Consuming 500 mg each day of cranberry dietary supplement may help reduce the risk of recurrent urinary tract infection (UTI) in healthy women. FDA has concluded that there is limited scientific evidence supporting this claim.”
“Consuming 500 mg [X capsules/tablets/soft gels] each day of [this identified cranberry dietary supplement] may help reduce the risk of recurrent urinary tract infection (UTI) in healthy women. FDA has concluded that there is limited scientific evidence supporting this claim.”

As a consequence, cranberry products are generally not recommended to use in place of proven treatment for a UTI.

### Urinary Tract Infection (UTI)

Escherichia coli (E. coli), a Gram-negative, facultative anaerobic, rod-shaped, coliform bacterium is commonly found in the lower intestine.

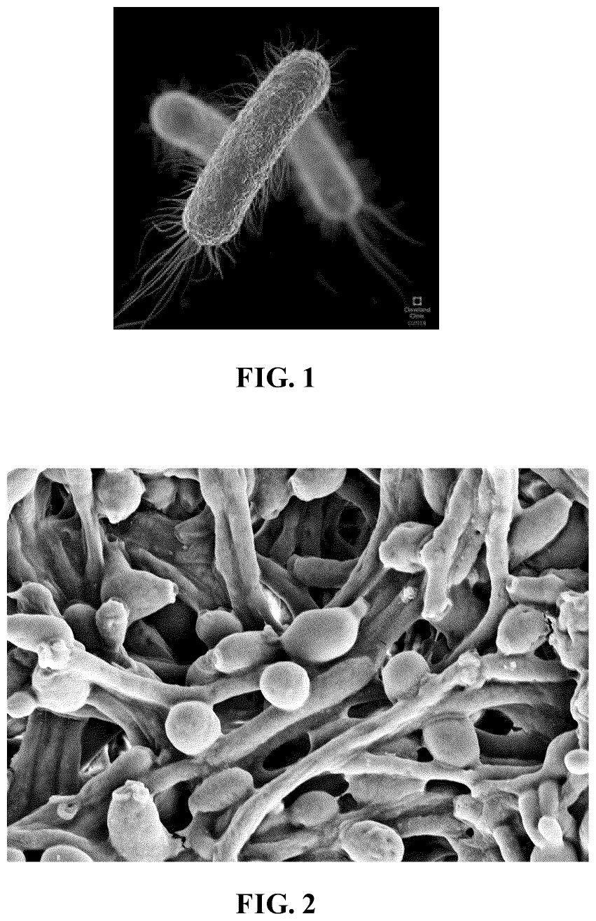

FIG. 1 is an image of E. coli cells.

The most well-known Shiga toxin-producing E. coli (STEC) is E. coli O157:H7 which can cause severe stomach cramps, bloody diarrhea and vomiting. Although healthy adults usually recover from infection with E. coli O157:H7 within a week, young children and older adults have a greater risk of developing a life-threatening form of kidney failure.

UTI is a common infection in any part of the urinary system (kidneys, ureters, bladder and urethra) affecting more than 3 million US cases per year. If an infection is limited to the bladder, it can be painful. However, serious health problems can result if a UTI spreads to the kidneys. UTIs don't always cause symptoms. When they do, they may include a strong urge to urinate that doesn't go away, a burning feeling when urinating, urinating often, and passing small amounts of urine, urine that looks cloudy, urine that appears red, bright pink or cola-colored—signs of blood in the urine, strong-smelling urine and pelvic pain. A UTI may result in more-specific symptoms depending on which part of the urinary tract is affected.

UTIs typically occur when bacteria enters the urinary tract through the urethra and begins to spread in the bladder. The urinary system is designed to keep out bacteria, however these defenses sometimes fail. When that happens, bacteria may take hold and grow into a full-blown infection in the urinary tract. In women, the urethra is close to the anus, and the urethral opening is close to the bladder. This makes it easier for bacteria around the anus to enter the urethra and to travel to the bladder. Infection of the urethra can happen when gastrointestinal bacteria spread from the anus to the urethra. Infection of the bladder is usually caused by Escherichia coli (E. coli), a type of bacteria commonly found in the gastrointestinal tract. An infection of the urethra can also be caused by sexually transmitted infections.

Antibiotics usually are the first treatment for urinary tract infections. Health status and the type of bacteria found in urine determine which medicine is used and how long a person needs to take it. However, any time antibiotics are taken, they can cause side effects, including include rashes, dizziness, nausea, diarrhea, and yeast infections.

### Vaginal Candidiasis (VC)

Candida albicans (C. albicans) are a gram-positive fungus that can take on a unicellular (yeast) or multicellular form. C. albicans is an opportunistic pathogenic yeast that is a common member of the human gut flora. It is detected in the gastrointestinal tract and mouth in 40-60% of healthy adults and is one of the few species of the genus Candida that causes the human infection candidiasis,

FIG. 2 is an image of an overgrowth of C. albicans.

Vaginitis is an inflammation of the vagina that can result in discharge, itching and pain. The cause is usually a change in the balance of vaginal bacteria or an infection. There are three common types of vaginitis: bacterial vaginosis (which results from an overgrowth of the bacteria naturally found in vagina), yeast infections (which are usually caused by a naturally occurring yeast Candida albicans) and trichomoniasis (which is caused by a parasite and is often sexually transmitted). Vaginitis signs and symptoms can include change in color, odor or amount of discharge from vagina, vaginal itching or irritation, pain during sex, painful urination and light vaginal bleeding or spotting. The characteristics of vaginal discharge might indicate the type of vaginitis. Treatment depends on the type of vaginitis.

Although vaginal candidiasis is often mild, some women can develop severe infections involving redness, swelling, and cracks in the wall of the vagina. A mortality rate of 40% has been reported for patients with systemic candidiasis due to C. albicans. By one estimate, invasive candidiasis contracted in a hospital causes 2,800 to 11,200 deaths per year in the US. However, about 20% of women normally have Candida in the vagina without having any symptoms.

In the United States, vaginal candidiasis is the second most common type of vaginal infection after bacterial vaginal infections. An estimated 1.4 million outpatient visits for vaginal candidiasis occur annually, however the number of vaginal candidiasis cases is unknown.”

Conventional treatment for yeast infections depends on the severity and frequency of infections. For mild to moderate symptoms and infrequent episodes, taking an antifungal medication for three to seven days will usually clear a yeast infection. Antifungal medications, which are available as creams, ointments, tablets and suppositories, include miconazole sold under the trade name MONISTAT and terconazole.

### EXPERIMENTAL EXAMPLES

### Example 1

A blend of five strains of Bifidobacteria and ten strains of Lactobacilli together with organic cranberry powder made from 100% cranberries was added to a comprising blend of organic red fruit and vegetable juices consisting of 100% organic watermelon juice, organic apple juice, organic pomegranate juice, organic beet juice and organic strawberry juice. Organic cranberry powder was added to the liquid blend of organic juices at a ratio of 500 mg per 2 fluid ounces before sterilization. Probiotics were added to the solution at a ratio of 27 billion CFUs per 2 fluid ounces to the sterilized blend of juices.

The probiotic medium was studied for its ability to inhibit three strains of E. coli O157:H7 and two strains of C. albicans.

### Active Ingredients

Species identification of probiotics was conducted by sequence analysis of the 16S rRNA genes while strain identification was conducted with pulse field gel electrophoresis (PFGE) analysis. The probiotics were shown to be sensitive to antibiotics and passed microbiological assays and heavy metal analysis. Probiotic strains were not genetically modified (GMO), were free from allergens, were considered safe with respect to bovine spongiform encephalopathy (BSE) and did not contain any colorant. The probiotics were also subjected to a series of in vitro tests that assess the gastrointestinal survival of probiotics (tolerance to hydrochloric acid—HCl), tolerance to bile salts, and resistance to gastrointestinal tract enzymes pepsin and pancreatin).

Organic cranberry powder was made from 100% cranberries. No sugar, preservative, flavor or color was added. The cranberry powder was subjected to physico-chemical analysis and microbiological analysis.

The probiotic blend is shown in the table below:

| Genus / Species | Strain |
|---|---|
| Bifidobacterium bifidum | SP 9 |
| Bifidobacterium breve | BBR8 |
| Bifidobacterium infantis | SP 37 |
| Bifidobacterium longum | SP 54 |
| Bifidobacterium animalis subsp. lactis | BLC1 |
| Lactobacillus acidophilus | LA1 |
| Lactobacillus brevis | SP 48 |
| Lactobacillus bulgaricus | LB2 |
| Lactobacillus casei | BGP 93 |
| Lactobacillus gasseri | LG050 |
| Lactobacillus paracasei | 101/37 |
| Lactobacillus plantarum | 14D |
| Lactobacillus reuteri | LR92 |
| Lactobacillus rhamnosus | SP 1 |
| Lactobacillus salivarius | SP2 |

### Probiotic Medium Preparation

Organic cranberry powder was added to the liquid blend of organic juices (500 mg per 2 fluid ounces) before sterilization. The blend of probiotics was added (27 billion CFU per 2 fluid ounces) to the sterilized blend of juices. Sensory evaluation of the formulation showed a pleasant taste. Every batch of this product is tested by an independent, FDA-registered accredited microbiology lab to confirm absence of pathogenic microorganisms.

The formulation (hereinafter referred to as “women's health formulation”) was packaged into two-ounce bottles for microbiology assays.

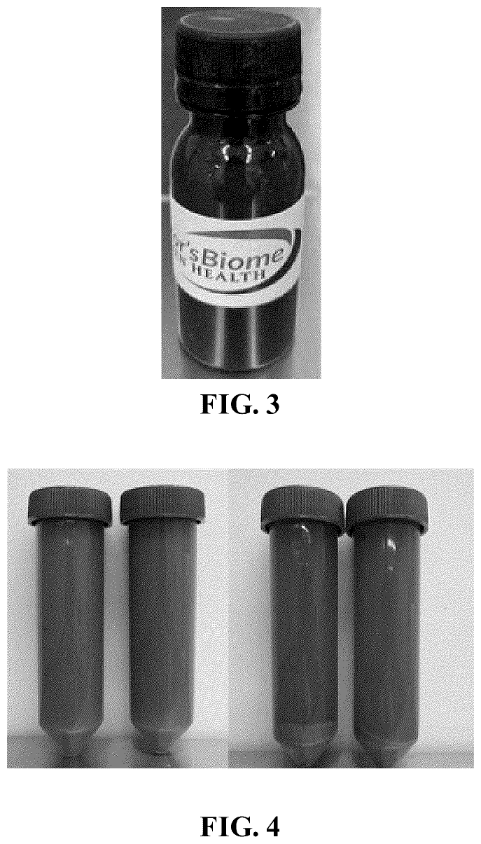

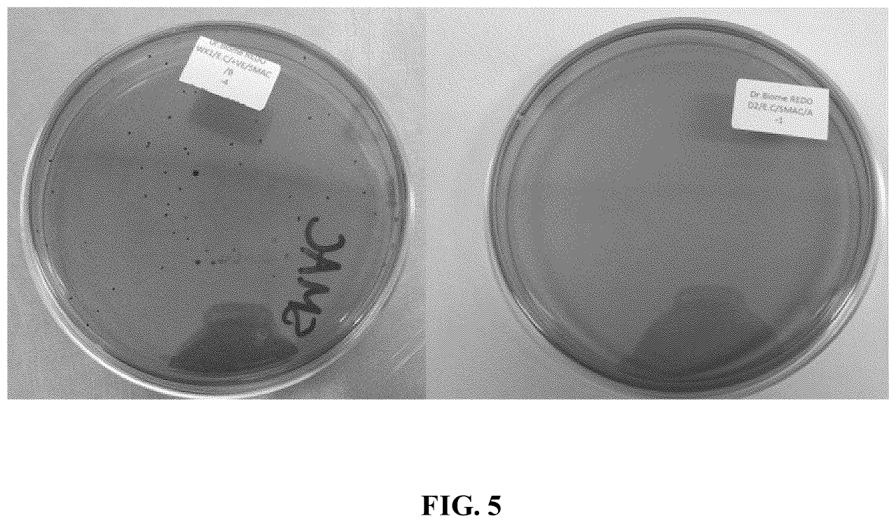

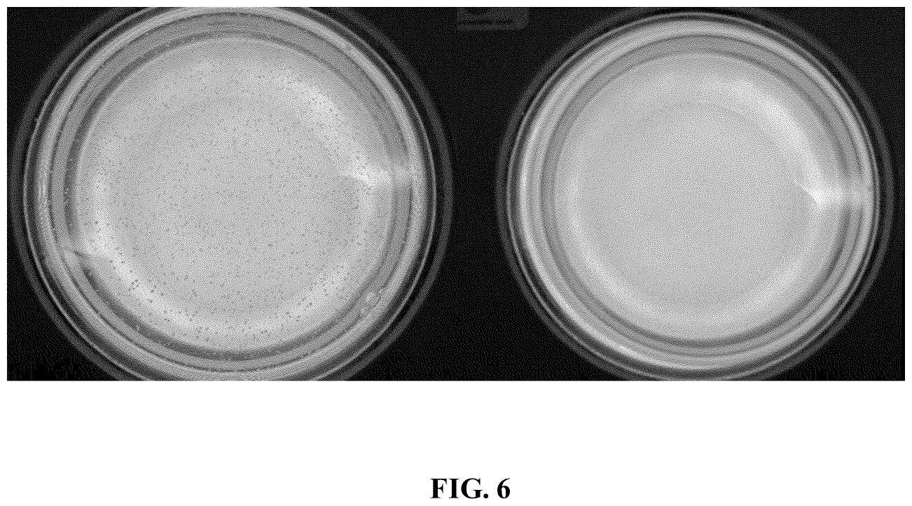

FIG. 3 shows samples of the women's health formulation. The samples were stored at refrigeration temperature.

### Pathogenic Organism Preparation

Three strains of E. coli O157:H7 and two strains of C. albicans were included in the study. The list of organisms and their sources of isolation is provided in the table below:

| Inoculum | | | |
|---|---|---|---|
| Cocktail | | Identification | |
| Group | Strain | Number¹ | Strain Information |
| E. coli | Escherichia | NFPA 4203 | Patient isolate |
| | coli O157:H7 | | |
| | Escherichia | NFPA 4206 | Human isolate, Univ. of |
| | coli O157:H7 | | Georgia |
| | Escherichia | NFPA 4210 | 1991 MA apple cider |
| | coli O157:H7 | | outbreak, patient isolate |
| Yeast | Candida albicans | ATCC 24433 | Nail infection |
| | Candida albicans | ATCC 14053 | Blood |

In order to prepare E. coli O157:H7 strains for this study, isolated colonies of each strain were individually sub-cultured into Tryptic Soy Broth (TSB) and incubated for 18±2 hours at 35° C. Subsequent growth was transferred to fresh TSB supplemented with 1% glucose (w/w) and incubated at 35° C. for 24±2 hours to facilitate acid adaptation. Following acid-adaptation, equivalent volumes of each culture were pooled, pelleted by centrifugation, washed once using 0.1% Peptone Water (PW), and then re-suspended in PW to form the E. coli O157:H7 inoculum cocktail. The inoculum cocktail was prepared the day of the study and stored at 4° C. when not in immediate use.

The Candida albicans strains were individually cultured in Potato Dextrose Broth (PDB) and incubated on a shaker at 25° C. for 2 days. Then, aliquots of the PDB were transferred to pre-poured acidified Potato Dextrose Agar (PDA) plates and incubated at 25° C. for 5 days. Following incubation, an equivalent number of plates per strain were flooded with 7 ml of PW and then gently scraped with a sterile spreader to collect surface growth. The resulting suspensions were combined to form the Candida albicans inoculum cocktail. The Candida albicans inoculum cocktail was then pelleted by centrifugation, washed once using PW, and then re-suspended in PW. The Candida albicans inoculum cocktail was prepared the day of the study and stored at 4° C. when not in immediate use.

### Preparation of Test Samples and Control Samples

The individual 2-ounce bottles of the women's health formulation were composited and homogenized to create one sample, and then centrifuged at 4,500 RPM for 15 minutes to prepare the “test sample.”

FIG. 4 shows an image of the women's health formulation before and after the centrifuge step.

The pH of the sample was measured prior to centrifugation (one replicate sample). To prepare the inoculated test samples, the women's health formulation was aliquoted into two portions and each portion was bulk-inoculated with one of the inoculum cocktails (either E. coli O157:H7 or C. albicans) at a target level of ˜4 log CFU/ml. Immediately following inoculation, the samples were mixed to ensure an even distribution of the inoculum.

Tryptic Soy broth (TSB) for E. coli 0l 57:H7 and Potato Dextrose broth (PDB) for C. albicans were used as the “control samples”, and they were inoculated in the same manner. The inoculated samples that were prepared for this study are summarized below:
Test Sample: women's health formulation centrifuged and inoculated with E. coli O157:H7
Test sample: women's health formulation centrifuged inoculated with C. albicans
Control sample: Tryptic Soy broth inoculated with E. coli O157:H7
Control sample: Potato Dextrose broth inoculated C. albicans

### Incubation of Test and Control Samples

The inoculated test samples and control samples were incubated at 37° C. for up to 5 days. The uninoculated samples (negative controls) were not included in the study due to limited product available.

The samples were evaluated according to the sampling scheme outlined in the table below:

| Sample | Incubation | | Replicates/Sampling |
|---|---|---|---|
| Description | Temperature | Sampling Times | Time |
| Test Samples | 37° C. | Days 0¹, 1, 2, 3, | 3 replicates² per |
| | | and 5 | organism |
| Control Samples | 37° C. | Days 0¹, 1, 2, 3, | 3 replicates² per |
| | | and 5 | organism |

¹Initial counts of organisms in test samples or control samples prior to the incubation at 37° C.
²1-ml sample removed from the test sample or control sample was considered to be one replicate.

### Evaluation of Samples

At each sampling time, the inoculated test samples and control samples were removed from the incubator and 3×1 ml aliquots of the samples were removed from the inoculated bulk sample, serially diluted with PW, and enumerated for the target organisms. Each 1 ml sample was considered to be one replicate sample. Appropriate dilutions were then pour-plated onto appropriate media. The plates were incubated under the appropriate conditions and then colonies were counted as the target organism based on characteristic colony morphology. No additional confirmations were done. The bulk samples were returned to the incubator.

A summary of the methods utilized are shown in the table below:

| | | Incubation Time and |
|---|---|---|
| Organism | Media | Temperature of the Plates |
| E. coli O157:H7 | Sorbitol MacConkey Agar | 1 day at 35° C. |
| | (SMAC) | |
| C. albicans | Potato Dextrose Agar | 5 days at 25° C. |
| | acidified with Tartaric Acid | |
| | (PDA) | |

### Results

All microbiological data were reported as log CFU/ml. The limit of detection by this plating method was 1.0 log CFU/ml. While on Day 0, E. coli population in the test sample is almost equal to its population in the control sample, E. coli growth is totally inhibited in the test sample from Day 1 to Day 5. On the other hand, E. coli in the control sample shows a typical growth curve for microorganisms (a drastic population increase peaking on Day 1 and then gradual decrease in population). Similarly, while on Day 0, C. albicans population in the test sample is almost equal to its population in the control sample, C. albicans growth in test sample is reduced by 70% on Day 1 and after that its growth is totally inhibited from Day 2 to Day 5. On the other hand, C. albicans in the control sample shows such an overwhelming growth that counts were outside countable range on highest dilution plated. Photographs of cultured plates show that colonies of E. coli recovered from the control sample at 1:10,000 dilution can be easily seen while no E. coli colony could be recovered from the test sample even at 1:10 dilution. This means total inhibition of E. coli by the dietary women's health formulation. Similarly, overwhelming number of colonies of C. albicans recovered from the control sample at 1:10,000 dilution can be seen while no C. albicans colony could be recovered from the test sample even at 1:10 dilution. Likewise, this means total inhibition of C. albicans by the dietary women's health formulation.

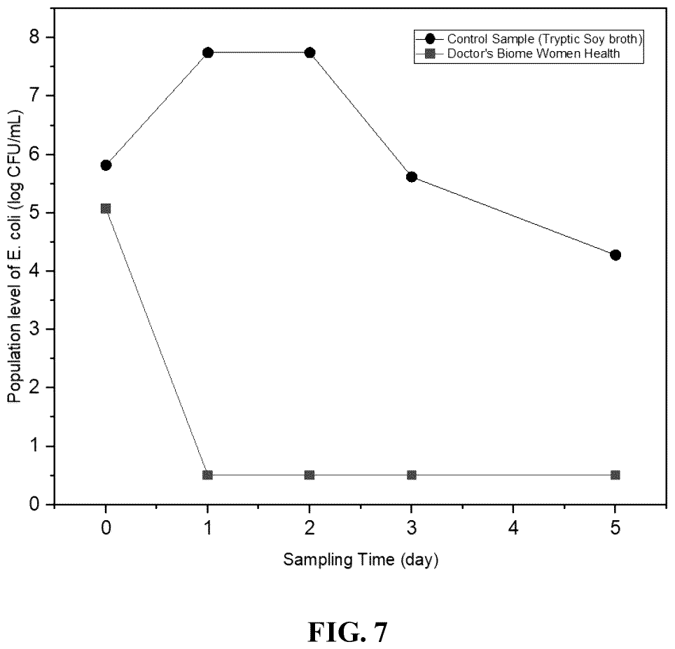

FIG. 7 is a graph of the population of E. coli O157:H7 recovered from a test sample and Tryptic Soy Broth (control samples) incubated at 37° C.

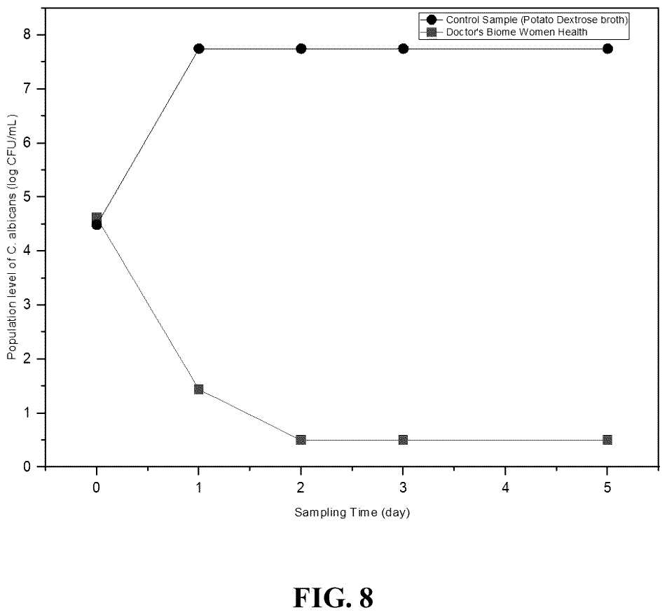

FIG. 8 is a graph of the population of C. albicans O157:H7 recovered from a test sample and Tryptic Soy Broth (control samples) incubated at 37° C.

The results are summarized in the tables below

Population of E. coli O157:H7 Recovered from the Test Sample and Tryptic Soy Broth (Control Samples) Incubated at 37° C.

| | | | Population Level of Target | |
|---|---|---|---|---|
| | | | Organism (log CFU/ml) | |
| | | | Doctor's | Control Samples |
| Target | Sampling | | Biome Women | (Tryptic Soy |
| Organisms | Times | Replicates | Health | broth) |
| E. coli | Day 0 | A | 5.45 | 5.86 |
| O157:H7 | | B | 5.25 | 5.79 |
| | | C | 4.52 | 5.81 |
| | | Avg ± SD¹ | 5.07 ± 0.49 | 5.82 ± 0.04 |
| | Day 1 | A | <1.00² | >7.75³ |
| | | B | <1.00² | >7.75³ |
| | | C | <1.00² | >7.75³ |
| | | Avg ± SD¹ | <1.00² | >7.75³ |
| | Day 2 | A | <1.00² | >7.75³ |
| | | B | <1.00² | >7.75³ |
| | | C | <1.00² | >7.75³ |
| | | Avg ± SD¹ | <1.00² | >7.75³ |
| | Day 3 | A | <1.00² | 5.60 |
| | | B | <1.00² | 5.59 |
| | | C | <1.00² | 5.66 |
| | | Avg ± SD¹ | <1.00² | 5.62 ± 0.04 |
| | Day 5 | A | <1.00² | 4.11 |
| | | B | <1.00² | 4.49 |
| | | C | <1.00² | 4.23 |
| | | Avg ± SD¹ | <1.00² | 4.28 ± 0.19 |

¹Avg ± SD: average ± standard deviation
²Below the limit of detection of 1.00 log CFU/ml
³Estimated count, counts were outside countable range on highest dilution plated.

Population of C. albicans Recovered from Doctor's Biome Women Health Product (Test Sample) and Tryptic Soy Broth (Control Samples) Incubated at 37° C.

| | | | Target Organism (log CFU/ml) | |
|---|---|---|---|---|
| | | | Doctor's | Control Samples |
| | | | Biome | (Potato |
| Target | Sampling | | Women | Dextrose |
| Organisms | Times | Replications | Health | Broth) |
| C. albicans | Day 0 | A | 4.61 | 4.46 |
| | | B | 4.59 | 4.45 |
| | | C | 4.66 | 4.56 |
| | | Avg ± SD¹ | 4.62 ± 0.04 | 4.49 ± 0.06 |
| | Day 1 | A | 1.48 | >7.75⁴ |
| | | B | 1.85 | >7.75⁴ |
| | | C | <1.00² | >7.75⁴ |
| | | Avg ± SD¹ | 1.44 ± 0.42³ | >7.75⁴ |
| | Day 2 | A | <1.00² | >7.75⁴ |
| | | B | <1.00² | >7.75⁴ |
| | | C | <1.00² | >7.75⁴ |
| | | Avg ± SD¹ | <1.00² | >7.75⁴ |
| | Day 3 | A | <1.00² | >7.75⁴ |
| | | B | <1.00² | >7.75⁴ |
| | | C | <1.00² | >7.75⁴ |
| | | Avg ± SD¹ | <1.00² | >7.75⁴ |
| | Day 5 | A | <1.00² | >7.75⁴ |
| | | B | <1.00² | >7.75⁴ |
| | | C | <1.00² | >7.75⁴ |
| | | Avg ± SD¹ | <1.00² | >7.75⁴ |

¹Avg ± SD: average ± standard deviation
²Below the limit of detection (1.00 log CFU/ml)
³For the purpose of calculation, the value <1.00 was treated as 1.00 to calculate average and standard deviation
⁴Estimated count, counts were outside countable range on highest dilution plated.

### Conclusion

The results of this in-vitro study indicate that E. coli O157:H7 and C. albicans (the microorganisms responsible for UTI and vaginal candidiasis) were totally inhibited by the women's health formulation to below the limit of detection of 1.0 log CFU/ml after 1 and 2 days of incubation (37° C.) respectively. In comparison, the population of these organisms grew in a normal way in the Tryptic Soy Broth and Potato Dextrose Broth under the same incubation conditions.

### Example 2

The novel women's health probiotic formulation of the invention, utilizing Bifidobacteria and Lactobacilli to formulate a novel probiotic together with cranberry powder in fruit and liquid juices, was provided to twenty four consumers. Their feedback, based on satisfaction and individual written comments, were collected. Consumer information and results are provided in the table below:

| Subject | | | Marital | Satisfaction | |
|---|---|---|---|---|---|
| # | Gender | Age | Status | Scale (1-5) | Comments |
| 1 | F | 65-74 | No | 4 | |
| 2 | F | 55-64 | | 5 | |
| 3 | F | 55-64 | Yes | 5 | |
| 4 | F | | | 5 | |
| 5 | F | 45-54 | Yes | 4 | |
| 6 | F | 65-74 | Yes | 4 | |
| 7 | F | 45-54 | Yes | 5 | |
| 8 | F | 55-64 | No | 5 | |
| 9 | F | 45-54 | No | 5 | I love it |
| 10 | F | 75-84 | No | 4 | Hoping the bottles are easier to open |
| 11 | F | 55-64 | Yes | 4 | |
| 12 | F | 45-54 | Yes | 4 | |
| 13 | M | 75-84 | Yes | 5 | My wife and I both feel better |
| 14 | F | | | 5 | I definitely can see a difference after |
| | | | | | two months |
| 15 | F | | | 5 | Just started this product noticed that |
| | | | | | I'm sleeping better and I've noticed my |
| | | | | | blood pressure is getting better. I intend |
| | | | | | to continue and hopefully there will be |
| | | | | | significant improvements. |
| 16 | F | | | 5 | Best probiotic I have ever taken! |
| 17 | F | 45-54 | | 5 | Very good product, I have been |
| | | | | | introduced this to all my patients and |
| | | | | | they love it |
| 18 | F | | | 5 | I'm feeling great since I started taking |
| | | | | | it's a good product |
| 19 | F | | | 5 | It was a great product. Really supported |
| | | | | | my gut and hair/nail growth. Would |
| | | | | | recommend, worth the price. |
| 20 | M | | Yes | 5 | I love this probiotic I think it has |
| | | | | | helped me tremendously I switched |
| | | | | | recently |
| 21 | M | | | 5 | Tastes delicious and I feel better than |
| | | | | | ever. Wanted to try this one even |
| | | | | | though I wasn't having any urinary |
| | | | | | tract issues. I take it an hour before my |
| | | | | | last of two daily meals and the Gut |
| | | | | | Health before my first meal. Both are |
| | | | | | excellent symbiotics. |
| 22 | F | | | 5 | Tastes great |
| 23 | F | | | 5 | Taste great |
| 24 | M | | | 5 | Great tasting drink! |

1. A probiotic formulation comprising:
a liquid medium comprising:
liquid fruit and vegetable juice;
cranberry powder added to the fruit and vegetable juice medium; and
a plurality of living probiotic bacterial strains of the genus Bifidobacterium and genus Lactobacillus, wherein the plurality of living probiotic bacterial strains of the genus Bifidobacterium include species B. bifidum, B. breve, B. lactis, B. longum, B. infantis and mixtures thereof and wherein the plurality of living probiotic bacterial strains of the genus Lactobacillus include species L. acidophilus, L. brevis, L. bulgaricus, L. casei, L. gasseri, L. paracasei, L. plantarum, L. reuteri, L. rhamnosus, L. salivarius and mixtures thereof;
wherein each living bacterial strain is present in the liquid medium in substantially a same percentage.

2. The probiotic formulation according to claim 1, wherein the liquid fruit and vegetable juice comprises watermelon juice, apple juice, pomegranate juice, beet juice and strawberry juice

3. The probiotic of claim 2, wherein the cranberry powder is present in the medium at a ratio of 500 mg per 2 fluid ounces of liquid fruit and vegetable juice.

4. The probiotic of claim 3, wherein the medium is sterilized prior to addition of the living probiotic bacterial strains.

5. The probiotic of claim 4, wherein the plurality of living probiotic bacterial strains is present in the medium at a ratio of 10-35 billion colony-forming units (CFU) per 2 fluid ounces of liquid fruit and vegetable juice.

6. A method of improving health of a woman with a urinary tract infection, the method comprising providing to a woman with an E. coli infection of the urinary tract a women's health probiotic formulation comprising:
a liquid medium comprising:
liquid fruit and vegetable juice; and
a plurality of living probiotic bacterial strains of the genus Bifidobacterium and genus Lactobacillus, wherein the plurality of living probiotic bacterial strains of the genus Bifidobacterium include species B. bifidum, B. breve, B. lactis, B. longum, B. infantis and mixtures thereof and wherein the plurality of living probiotic bacterial strains of the genus Lactobacillus include species L. acidophilus, L. brevis, L. bulgaricus, L. casei, L. gasseri, L. paracasei, L. plantarum, L. reuteri, L. rhamnosus, L. salivarius and mixtures thereof;
wherein each living bacterial strain is present in the liquid medium in substantially a same percentage.

7. The method of claim 6, wherein the liquid fruit and vegetable juice comprises watermelon juice, apple juice, pomegranate juice, beet juice and strawberry juice

8. The method of claim 7, further comprising cranberry powder added to the fruit and vegetable juice medium, wherein the cranberry powder is present in the medium at a ratio of 500 mg per 2 fluid ounces of liquid fruit and vegetable juice.

9. The method of claim 8, wherein the medium is sterilized prior to addition of the living probiotic bacterial strains.

10. The method of claim 9, wherein the plurality of living probiotic bacterial strains is present in the medium at a ratio of between 10-35 billion colony-forming units (CFU) per 2 fluid ounces of liquid fruit and vegetable juice.

11. A method of improving health of a woman with a yeast infection, the method comprising providing to a woman with an C. albicans infection a probiotic formulation comprising:
a liquid medium comprising:
liquid fruit and vegetable juice; and
a plurality of living probiotic bacterial strains of the genus Bifidobacterium and genus Lactobacillus, wherein the plurality of living probiotic bacterial strains of the genus Bifidobacterium include species B. bifidum, B. breve, B. lactis, B. longum, B. infantis and mixtures thereof and wherein the plurality of living probiotic bacterial strains of the genus Lactobacillus include species L. acidophilus, L. brevis, L. bulgaricus, L. casei, L. gasseri, L. paracasei, L. plantarum, L. reuteri, L. rhamnosus, L. salivarius and mixtures thereof;
wherein each living bacterial strain is present in the liquid medium in substantially a same percentage.

12. The method of claim 11, wherein the liquid fruit and vegetable juice comprises watermelon juice, apple juice, pomegranate juice, beet juice and strawberry juice

13. The method of claim 12, further comprising cranberry powder added to the fruit and vegetable juice medium, wherein the cranberry powder is present in the medium at a ratio of 500 mg per 2 fluid ounces of liquid fruit and vegetable juice.

14. The method of claim 13, wherein the medium is sterilized prior to addition of the living probiotic bacterial strains.

15. The method of claim 14, wherein the plurality of living probiotic bacterial strains is present in the medium at a ratio of between 25-30 billion colony-forming units (CFU) per 2 fluid ounces of liquid fruit and vegetable juice.
